(19) United States
(12) Patent Application Publication
(10) Pub. No.: US 2026/0315003 A1
(43) Pub. Date: Oct. 15, 2026

(54) BIOLOGICAL INFORMATION MEASUREMENT DEVICE, BIOLOGICAL INFORMATION MEASUREMENT METHOD, AND RECORDING MEDIUM

(71) Applicant: OMRON HEALTHCARE Co., Ltd., Kyoto (JP)

(72) Inventors: Tetsuho ONO, Kyoto (JP); Chiaki EBISU, Kyoto (JP); Tatsuya KOBAYASHI, Kyoto (JP); Mika EZOE, Kyoto (JP); Takanobu YAMAUCHI, Kyoto (JP); Yotaro NISHIO, Kyoto (JP); Yuki SAKAGUCHI, Kyoto (JP); Iwanori NAMBA, Kyoto (JP); Kenichi HINUMA, Kyoto (JP)

(21) Appl. No.: 19/683,289

(22) Filed: May 20, 2026

Related U.S. Application Data
(63) Continuation of application No. PCT/JP2025/000864, filed on Jan. 14, 2025.

(30) Foreign Application Priority Data
Mar. 13, 2024 (JP) 2024-038977

(51) Int. Cl. A61B 5/00; A61B 5/02

(52) U.S. Cl. CPC A61B 5/6829; A61B 5/02; A61B 5/4023; A61B 5/4872

(57) ABSTRACT

To improve usability in measurement of biological information. A biological information measurement device having a placement surface on which a subject can step includes a detection unit configured to detect a stepping manner selected by the subject from among a plurality of stepping manners for the placement surface, and a measurement unit configured to measure biological information of the subject, wherein an operation of the measurement unit differs depending on the stepping manner detected by the detection unit.

### CROSS-REFERENCE TO RELATED APPLICATIONS

This application is the U.S. national stage application filed pursuant to 35 U.S.C. 365(c) and 120 as a continuation of International Patent Application No. PCT/JP2025/000864, filed Jan. 14, 2025, which application claims priority to Japanese Patent Application No. 2024-038977, filed Mar. 13, 2024, which applications are incorporated herein by reference in their entireties.

### TECHNICAL FIELD

The present invention relates to a biological information measurement device, a biological information measurement method, and a recording medium or program.

### BACKGROUND ART

Patent Document 1 discloses a technology that enables a process of measuring body fat and a process of measuring posture balance of the body to be performed simultaneously by a single device. Patent Document 2 discloses a technology that improves measurement accuracy of body fat in a separate type body fat meter in which a weight measurement unit and an electrode unit are separated. Patent Document 3 discloses a technology for measuring characteristics of a user's cardiovascular system. Patent Document 4 discloses a visceral fat measuring device that displays visceral fat information and includes a setting registration key for setting and registering unique biological information about a body and time, a personal key for calling and measuring the unique biological information about the body, and a weight dedicated key for measuring only weight.

### CITATION LIST

Patent Document 1: JP 2003-339671 A

Patent Document 2: JP H11-244252 A

Patent Document 3: JP 2014-507213 A

Patent Document 4: JP 2002-238870 A

### SUMMARY OF INVENTION

### Technical Problem

A general body composition meter includes a load cell and an electrode unit that comes into contact with a human body, and performs weight measurement and bioelectrical impedance measurement using the load cell and the electrode unit, thereby displaying results related to weight or various body compositions. The measurement time is several seconds, and the measurement results are not significantly affected by body motion of a subject, such as movement of a head to confirm a measurement progress state displayed at feet during measurement. On the other hand, when functions for performing ballistocardiogram (BCG) measurement using a load cell and impedance pulse wave (IPG) measurement using an electrode and for presenting cardiovascular condition information to a user are added to a body composition meter, body motion caused by movement of the head or arm during measurement significantly affects measurement results. As a result, measurement time may be prolonged, or remeasurement may be required due to a measurement error. In addition, in typical configurations, a series of data collection operations in a body composition meter is performed simultaneously in parallel and results for all measurement items are displayed in order after a predetermined measurement time has elapsed. Thus, as the number of measurement items increases, considerable measurement time is required when the time for displaying the results is included. However, when a body composition meter is shared by family members, a management target may be only a weight value, and a measurement target item may be limited to weight.

In Patent Document 1, in a first operation, a subject steps on a weight scale, places the left foot on a left foot electrode attached to a step, places the right foot on the same step as the left foot, and measures weight, and in a second operation, the subject spreads the legs, moves the right foot from the step to a right foot electrode attached to a support base, and measures impedance (see paragraphs [0013], [0014], and the like of Patent Document 1). Therefore, in Patent Document 1, it is necessary to move the foot by a large amount during the measurement, and the measurement is not completed with the positions of the feet at the time of stepping on the weight scale maintained, resulting in reduced usability. In Patent Document 2, it is standard that two electrode units are placed on both left and right sides of a weight measurement unit and used, a subject steps on the central weight measurement unit to acquire weight data, and the subject steps on the two electrode units to acquire impedance data (see paragraph [0025] of Patent Document 2). Therefore, in Patent Document 2, it is necessary to move the feet by a large amount during the measurement, and the measurement is not completed with the positions of the feet at the time of stepping on the weight measurement unit or the electrode units maintained, resulting in reduced usability. For example, when a body composition meter is provided with a weight dedicated key as in Patent Document 4, the weight dedicated key needs to be added, a key operation before measurement is cumbersome, and usability is reduced.

The present invention has been made in view of the above circumstances, and an object thereof is to improve usability in measurement of biological information.

### Solution To Problem

According to an aspect of the present invention, there is provided a biological information measurement device having a placement surface on which a subject is to step, the biological information measurement device including: a detection unit configured to detect a stepping manner selected by the subject from among a plurality of stepping manners for the placement surface; and a measurement unit configured to measure biological information of the subject, wherein an operation of the measurement unit differs depending on the stepping manner detected by the detection unit. The detection unit detects the stepping manner selected by the subject from among the plurality of stepping manners for the placement surface of the biological information measurement device. The operation of the measurement unit differs depending on the stepping manner detected by the detection unit. The measurement unit performs different operations depending on the stepping manner detected by the detection unit, and thus usability in measurement of biological information is improved.

The detection unit detects the stepping manner selected by the subject by detecting an order in which the left foot and the right foot of the subject are placed on the placement surface. The operation of the measurement unit differs depending on the order in which the left foot and the right foot of the subject are placed on the placement surface of the biological information measurement device. The measurement unit performs different operations depending on the order in which the left foot and the right foot of the subject are placed on the placement surface of the biological information measurement device, and thus usability in measurement of biological information is improved.

The detection unit detects the stepping manner selected by the subject by detecting a direction in which the subject faces at the time when the subject steps on the placement surface. The operation of the measurement unit differs depending on the direction in which the subject faces at the time when the subject steps on the placement surface of the biological information measurement device. The measurement unit performs different operations depending on the direction in which the subject faces at the time when the subject steps on the placement surface of the biological information measurement device, and thus usability in measurement of biological information is improved.

The detection unit detects the stepping manner selected by the subject by detecting an arrangement of the feet of the subject on the placement surface of the biological information measurement device. The operation of the measurement unit differs depending on the arrangement of the feet of the subject on the placement surface of the biological information measurement device. The measurement unit performs different operations depending on the arrangement of the feet of the subject on the placement surface of the biological information measurement device, and thus usability in measurement of biological information is improved.

A time for measurement of the biological information by the measurement unit differs depending on the stepping manner detected by the detection unit. A measurement item for measurement of the biological information by the measurement unit differs depending on the stepping manner detected by the detection unit. A recording target of a measurement result of the biological information differs depending on the stepping manner detected by the detection unit.

The biological information measurement device includes a first electrode being to come into contact with a sole of one of both feet of the subject and a second electrode being to come into contact with a sole of the other of both feet of the subject, and the number of the first electrodes differs from the number of the second electrodes. This makes it possible to guide the subject to intentionally place one or the other of both feet of the subject on the placement surface of the biological information measurement device.

The biological information measurement device includes a first electrode being to come into contact with a sole of one of both feet of the subject and a second electrode being to come into contact with a sole of the other of both feet of the subject, and a shape of the first electrode differs from a shape of the second electrode. This makes it possible to guide the subject to intentionally place one or the other of both feet of the subject on the placement surface of the biological information measurement device.

Note that the present invention can also be understood as a biological information measurement method in which a computer executes at least part of the above processing, a biological information measurement method including at least part of the above processing, a program for causing a computer to execute at least part of the above processing, or a computer-readable recording medium on which such a program is non-transitorily recorded. The configurations and processes described above can be combined with one another to constitute the present invention unless the combination leads to technical contradiction.

### Advantageous Effects of Invention

According to the present invention, usability in measurement of biological information can be improved.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a diagram illustrating a schematic configuration of a biological information measurement device according to an embodiment.

FIG. 2 is a block diagram illustrating a configuration of the biological information measurement device according to the embodiment.

FIG. 3 is a diagram illustrating an arrangement of a plurality of load cells.

FIG. 4 is a diagram illustrating an arrangement of the plurality of load cells and an acceleration sensor.

FIG. 5 is a block diagram illustrating a functional configuration of a control unit.

FIG. 6 is a diagram illustrating the relationship between a BCG waveform, a foot IPG waveform, and a PTT.

FIG. 7 is a diagram illustrating an arrangement of photoelectric sensors.

FIG. 8 is a diagram illustrating a schematic configuration of a biological information measurement device according to an embodiment.

FIG. 9 is a diagram illustrating a schematic configuration of a biological information measurement device according to an embodiment.

FIG. 10 is a diagram illustrating a schematic configuration of a biological information measurement device according to an embodiment.

FIG. 11 is a diagram illustrating a schematic configuration of a biological information measurement device according to an embodiment.

FIG. 12 is a diagram illustrating a schematic configuration of a biological information measurement device according to an embodiment.

FIG. 13 is a diagram illustrating a schematic configuration of a biological information measurement device according to an embodiment.

FIG. 14 is a diagram illustrating a schematic configuration of a biological information measurement device according to an embodiment.

FIG. 15 is a diagram illustrating a schematic configuration of a biological information measurement device according to an embodiment.

FIG. 16 is a diagram illustrating a schematic configuration of a biological information measurement device according to an embodiment.

FIG. 17 is a diagram illustrating a schematic configuration of a biological information measurement device according to an embodiment.

FIG. 18 is a diagram illustrating a schematic configuration of the biological information measurement device according to the embodiment.

FIG. 19 is a diagram illustrating a schematic configuration of the biological information measurement device according to the embodiment.

FIG. 20 is a diagram illustrating a schematic configuration of a biological information measurement device according to an embodiment.

FIG. 21 is a diagram illustrating a schematic configuration of a biological information measurement device according to an embodiment.

### DESCRIPTION OF EMBODIMENTS

Embodiments will be described below with reference to the drawings. The embodiments described below are an aspect of the present application and do not limit the scope of the present application.

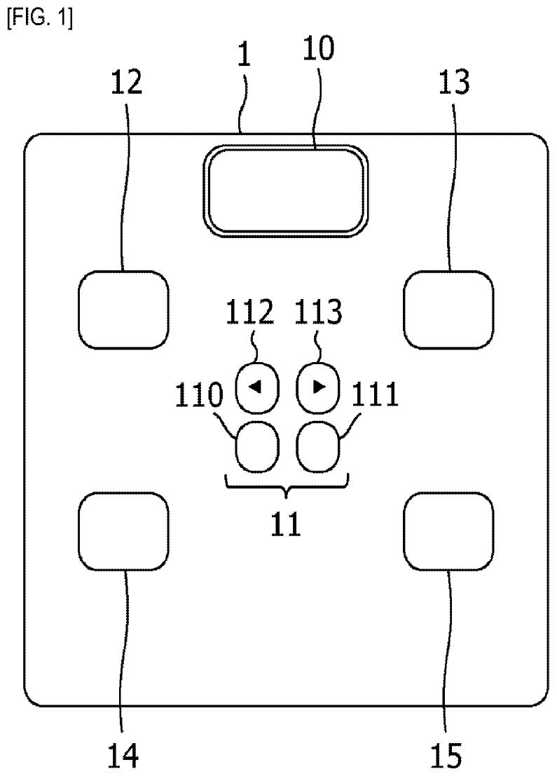

FIG. 1 is a diagram illustrating a schematic configuration of a biological information measurement device 1 according to an embodiment. The biological information measurement device 1 includes a display unit 10, an operation unit 11, and electrodes 12 to 15. The biological information measurement device 1 has a front surface (placement surface) on which a subject such as a user can step. The electrodes 12 and 14 are arranged on the front surface of the biological information measurement device 1 such that, when the subject steps on the front surface of the biological information measurement device 1, a front end side (toe side) of a sole of the left foot comes into contact with the electrode 12 and a rear end side (heel side) of the sole of the left foot comes into contact with the electrode 14. The electrodes 13 and 15 are arranged on the front surface of the biological information measurement device 1 such that, when the subject steps on the front surface of the biological information measurement device 1, a front end side (toe side) of a sole of the right foot comes into contact with the electrode 13 and a rear end side (heel side) of the sole of the right foot comes into contact with the electrode 15.

The display unit 10 is provided on the front surface of the biological information measurement device 1. The operation unit 11 is provided at a center portion of the front surface of the biological information measurement device 1. For example, the subject steps on the front surface of the biological information measurement device 1 such that the front end side and the rear end side of the sole of the left foot of the subject come into contact with the electrodes 12 and 14, respectively, and the front end side and the rear end side of the sole of the right foot of the subject come into contact with the electrodes 13 and 15, respectively.

The operation unit 11 includes switches 110 to 113. The switch 110 is a switch that receives input of an instruction to turn on or off a power source of the biological information measurement device 1 and an instruction to start and end measurement. The switch 111 is a switch that receives an instruction to display, on the display unit 10, various types of data stored in the biological information measurement device 1. The switches 112 and 113 are switches that receive selection and the like of various data displayed on the display unit 10. In addition, the switches 111 to 113 are used to receive data input.

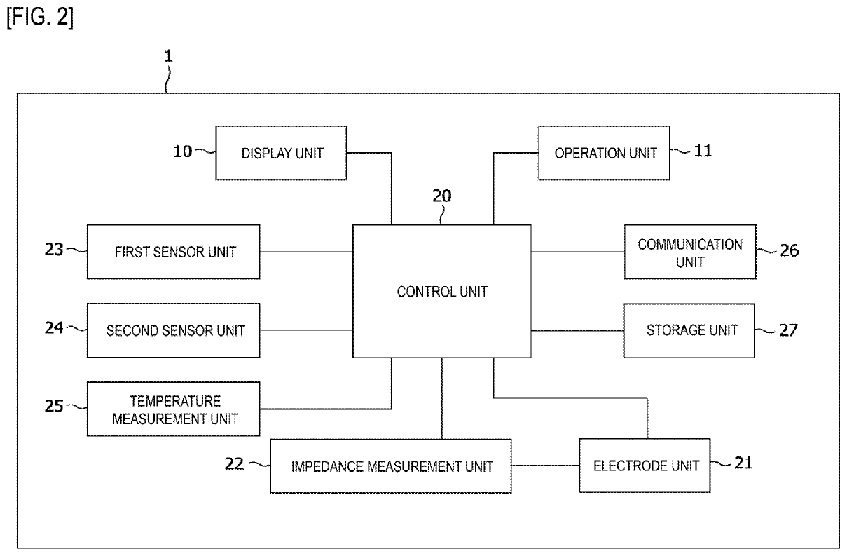

FIG. 2 is a block diagram illustrating a configuration of the biological information measurement device 1 according to the embodiment. The biological information measurement device 1 includes the display unit 10, the operation unit 11, a control unit 20, an electrode unit 21, an impedance measurement unit 22, a first sensor unit 23, a second sensor unit 24, a temperature measurement unit 25, a communication unit 26, and a storage unit 27. For example, a liquid crystal display panel such as a liquid crystal display (LCD), an electroluminescence (EL) display, or the like is used for the display unit 10. The display unit 10 displays measurement results as well as various types of information and data. The operation unit 11 receives an operation from the subject.

The control unit 20 is a control device (controller) that controls the entire operation of the biological information measurement device 1. The control unit 20 may be configured by a dedicated device or may be configured by a general-purpose computer. The control unit 20 includes hardware resources such as a central processing unit (CPU), a memory, and a storage. The memory may be a random access memory (RAM). The storage may be a non-volatile storage device such as a read only memory (ROM) or a flash memory. The functions of the control unit 20 as the processing units (functional units) are realized by loading a program stored in the storage onto a memory and executing the program by a processor. Note that the configuration of the control unit 20 is not limited to the above. For example, the functions of the control unit 20 may be entirely or partially configured by a circuit such as an ASIC or an FPGA, or the functions of the control unit 20 may be entirely or partially executed by a cloud server or another device.

The electrode unit 21 includes the electrodes 12 to 15. The electrodes 12 to 15 are used to apply a constant current to the body of the subject so as to measure bioelectrical impedance of the subject, and to measure a voltage when the current is applied. The electrode unit 21 may include other electrodes different from the electrodes 12 to 15. The impedance measurement unit 22 measures bioelectrical impedance of the subject, based on respective values of a current applied to the body of the subject and a voltage measured during current application. The impedance measurement unit 22 outputs the bioelectrical impedance of the subject to the control unit 20. The impedance measurement unit 22 may output the bioelectrical impedance of the subject as an impedance pulse wave.

The first sensor unit 23 includes a photoelectric sensor. The first sensor unit 23 outputs a measurement value measured by the photoelectric sensor to the control unit 20. The second sensor unit 24 has a plurality of load cells (strain gauges). The second sensor unit 24 outputs measurement values measured by the plurality of load cells to the control unit 20.

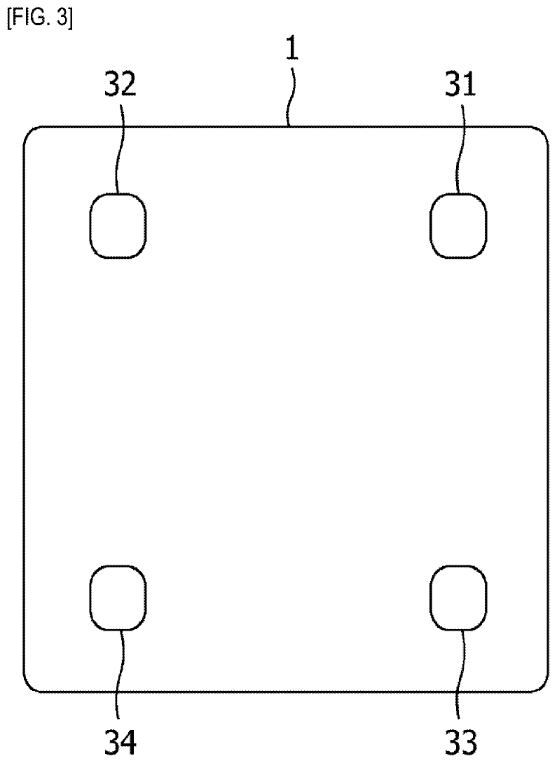

FIG. 3 is a diagram illustrating an arrangement of a plurality of load cells. As illustrated in FIG. 3, load cells 31 to 34 are disposed on a back side surface of the biological information measurement device 1. The load cells 31 to 34 are respectively disposed at four corners on the back side surface of the biological information measurement device 1. In consideration of the influence of body motion of the subject, the load cells 31 to 34 are preferably arranged so that load is evenly applied to the load cells 31 to 34. In a thickness direction of the biological information measurement device 1, the electrode 12 and the load cell 31 may overlap, the electrode 13 and the load cell 32 may overlap, the electrode 14 and the load cell 33 may overlap, and the electrode 15 and the load cell 34 may overlap. The second sensor unit 24 may include an acceleration sensor. The second sensor unit 24 may output a measurement value measured by the acceleration sensor to the control unit 20.

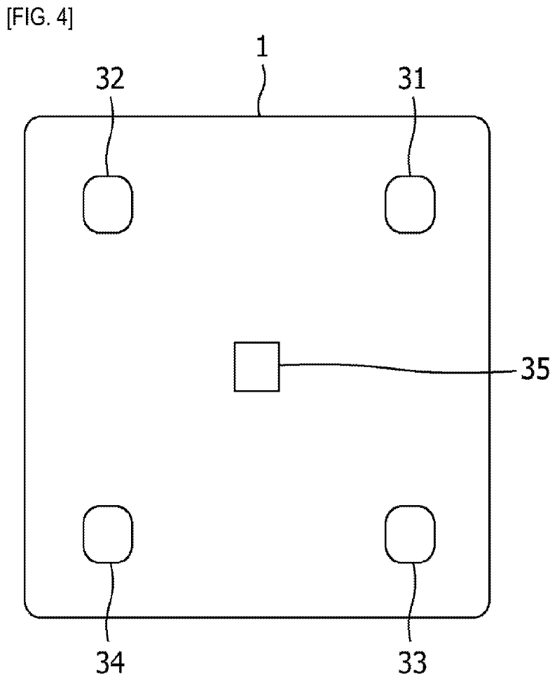

FIG. 4 is a diagram illustrating an arrangement of the plurality of load cells and an acceleration sensor. As illustrated in FIG. 4, the load cells 31 to 34 and an acceleration sensor 35 are disposed on the back side surface of the biological information measurement device 1. The load cells 31 to 34 are respectively disposed at four corners on the back side surface of the biological information measurement device 1. In consideration of the influence of body motion of the subject, the load cells 31 to 34 are preferably arranged so that load is evenly applied to the load cells 31 to 34. In the thickness direction of the biological information measurement device 1, the electrode 12 and the load cell 31 may overlap, the electrode 13 and the load cell 32 may overlap, the electrode 14 and the load cell 33 may overlap, and the electrode 15 and the load cell 34 may overlap. The acceleration sensor 35 is disposed at a center portion of the back side surface of the biological information measurement device 1.

The temperature measurement unit 25 measures temperatures of the soles of the feet of the subject stepping on the front surface of the biological information measurement device 1. The temperature measurement unit 25 includes a temperature sensor. The temperature sensor is disposed on the front surface of the biological information measurement device 1. The temperature sensor may be disposed in the vicinity of the electrodes 12 to 15.

The communication unit 26 is an interface for performing wired or wireless communication. The communication unit 26 communicates with an information processing device of the subject. The information processing device is, for example, a portable terminal, a smartphone, a tablet terminal, a personal computer, or the like.

The storage unit 27 stores information and data. The storage unit 27 may include at least one of a memory such as a RAM or a storage. The storage may be a non-volatile storage device such as a ROM or a flash memory. The storage unit 27 stores various types of information such as personal data of the subject and measurement results of the biological information measurement device 1. The personal data is used when calculating body composition of the subject. The personal data includes at least the height and weight of the subject, and may further include the age and sex of the subject, as well as other types of information. The weight of the subject is measured when the subject steps on the front surface of the biological information measurement device 1. When the subject operates the operation unit 11, the height, age, sex, and other types of information of the subject are input to the biological information measurement device 1.

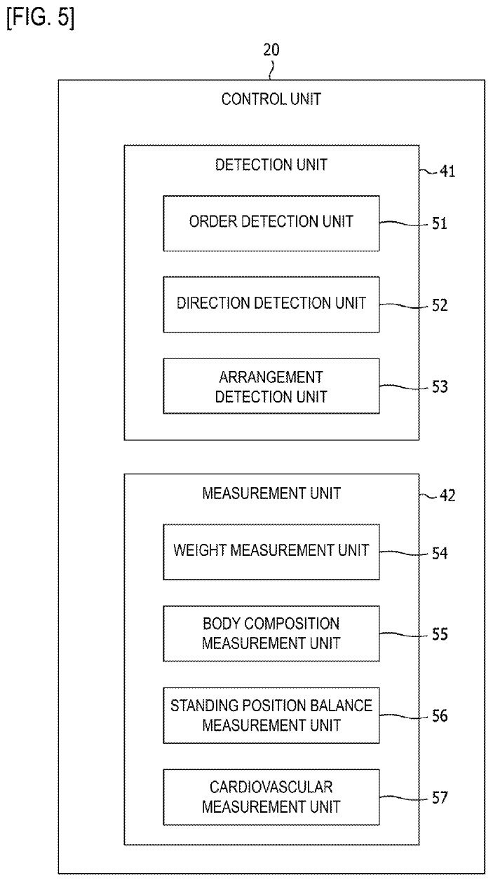

FIG. 5 is a block diagram illustrating a functional configuration of the control unit 20. The control unit 20 includes a detection unit 41 and a measurement unit 42. The detection unit 41 includes an order detection unit 51, a direction detection unit 52, and an arrangement detection unit 53. The measurement unit 42 includes a weight measurement unit 54, a body composition measurement unit 55, a standing position balance measurement unit 56, and a cardiovascular measurement unit 57.

The detection unit 41 detects a stepping manner selected by the subject from among a plurality of stepping manners for the front surface of the biological information measurement device 1. The measurement unit 42 measures biological information of the subject. The order detection unit 51 detects the stepping manner selected by the subject by detecting an order in which the left foot and the right foot of the subject are placed on the front surface of the biological information measurement device 1, based on the measurement value output from at least one of the electrode unit 21, the first sensor unit 23, and the second sensor unit 24.

The direction detection unit 52 detects the stepping manner selected by the subject by detecting a direction in which the subject faces at the time when the subject steps on the front surface of the biological information measurement device 1, based on the measurement value output from at least one of the electrode unit 21, the first sensor unit 23, and the second sensor unit 24. The arrangement detection unit 53 detects the stepping manner selected by the subject by detecting an arrangement of the feet of the subject on the front surface of the biological information measurement device 1, based on the measurement value output from at least one of the electrode unit 21 and the first sensor unit 23.

The weight measurement unit 54 measures a weight of the subject stepping on the front surface of the biological information measurement device 1, based on the measurement value output from the second sensor unit 24. The body composition measurement unit 55 measures body composition of the subject stepping on the front surface of the biological information measurement device 1, based on the personal data of the subject, the bioelectrical impedance output from the impedance measurement unit 22, and the weight of the subject, according to a predetermined algorithm. The body composition is an indicator indicating a percentage or an amount of components constituting the body. Examples of the body composition include, but are not limited to, body fat percentage, visceral fat level, skeletal muscle percentage, basal metabolism, body age, BMI, muscle percentage, muscle mass, body fat mass, bone mass, water content, and the like. The personal data of the subject includes the weight and the bioelectrical impedance measured in the past. The body composition measurement unit 55 may identify the subject by comparing the weight and the bioelectrical impedance measured in the past with the weight and the bioelectrical impedance currently measured. The body composition measurement unit 55 acquires the personal data of the identified subject from the storage unit 27.

The standing position balance measurement unit 56 measures the balance of the subject in a standing position (standing position balance) stepping on the front surface of the biological information measurement device 1, based on the measurement value output from the second sensor unit 24. The standing position balance measurement unit 56 may measure the standing position balance of the subject by calculating the position of the center of gravity of the subject in the standing position. The standing position balance measurement unit 56 may measure the standing position balance of the subject according to a predetermined algorithm.

The cardiovascular measurement unit 57 measures a cardiovascular condition of the subject stepping on the front surface of the biological information measurement device 1, based on the measurement value output from the second sensor unit 24 and the bioelectrical impedance output from the impedance measurement unit 22, according to a predetermined algorithm. The cardiovascular measurement unit 57 measures ballistocardiogram (BCG), which is vibration caused by the blood being pumped out from the heart, based on the measurement value output from the second sensor unit 24. The cardiovascular measurement unit 57 measures the leg impedance pulse wave (IPG) and the foot impedance pulse wave (IPG), based on the bioelectrical impedance output from the impedance measurement unit 22. The leg IPG is an indicator indicating a change in the amount of blood in the blood vessel of the leg. The foot IPG is an indicator indicating a change in the amount of blood in the blood vessel of the foot. The cardiovascular measurement unit 57 creates an average waveform of the foot IPG (foot IPG waveform) with reference to the leg IPG. The cardiovascular measurement unit 57 calculates a pulse transit time (PTT) from the BCG waveform and a feature point of the foot IPG waveform.

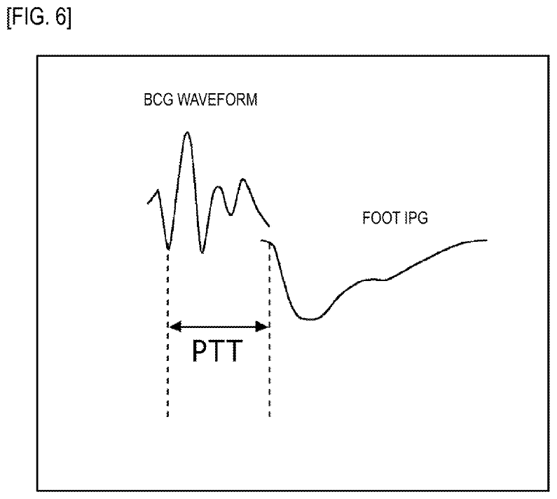

FIG. 6 is a diagram illustrating the relationship between the BCG waveform, the foot IPG waveform, and the PTT. As illustrated in FIG. 6, the pulse transit time (PTT) can be calculated by obtaining a (time) difference between a reference point of the BCG waveform and the feature point of the foot IPG waveform. The cardiovascular measurement unit 57 calculates a pulse wave velocity (PWV) by dividing a measurement section distance by the PTT. Since the value of the PWV increases as the blood vessel becomes harder, the PWV is used as an indicator indicating hardness of the blood vessel. The cardiovascular measurement unit 57 converts the pulse wave velocity (PWV) into an indicator (cardiovascular indicator) indicating a cardiac function or a state of a blood vessel, based on a predetermined algorithm, and calculates the cardiovascular indicator. In order to measure the cardiovascular condition of the subject, it is necessary to maintain for about 20 to 30 seconds, a state in which there is no body motion of the subject or a state in which the body motion of the subject is small. Therefore, the cardiovascular measurement unit 57 starts measuring the cardiovascular condition of the subject with the subject making no body motion or making a small body motion.

The detection unit 41 detects a stepping manner selected by the subject from among a plurality of stepping manners for the front surface of the biological information measurement device 1. The operation of the measurement unit 42 differs depending on the stepping manner detected by the detection unit 41. The measurement unit 42 performs different operations depending on the stepping manner detected by the detection unit 41, and thus usability in measurement of biological information is improved.

The order detection unit 51 detects the stepping manner selected by the subject by detecting an order in which the left foot and the right foot of the subject are placed on the front surface of the biological information measurement device 1. Therefore, the operation of the measurement unit 42 differs depending on the order in which the left foot and the right foot of the subject are placed on the front surface of the biological information measurement device 1. The measurement unit 42 performs different operations depending on the order in which the left foot and the right foot of the subject are placed on the front surface of the biological information measurement device 1, and thus usability in measurement of biological information is improved.

The direction detection unit 52 detects the stepping manner selected by the subject by detecting a direction in which the subject faces at the time when the subject steps on the front surface of the biological information measurement device 1. Therefore, the operation of the measurement unit 42 differs depending on the direction in which the subject faces at the time when the subject steps on the front surface of the biological information measurement device 1. The measurement unit 42 performs different operations depending on the direction in which the subject faces at the time when the subject steps on the front surface of the biological information measurement device 1, and thus usability in measurement of biological information is improved.

The arrangement detection unit 53 detects the stepping manner selected by the subject by detecting an arrangement of the feet of the subject on the front surface of the biological information measurement device 1. Therefore, the operation of the measurement unit 42 differs depending on the arrangement of the feet of the subject on the front surface of the biological information measurement device 1. The measurement unit 42 performs different operations depending on the arrangement of the feet of the subject on the front surface of the biological information measurement device 1, and thus usability in measurement of biological information is improved.

Next, detection of the stepping manner selected by the subject will be described. The manner in which the subject steps on the front surface of the biological information measurement device 1 includes an order in which the left foot and the right foot of the subject are placed on the front surface of the biological information measurement device 1. The manner in which the subject steps on the front surface of the biological information measurement device 1 includes a direction in which the subject steps on the biological information measurement device 1, that is, a direction in which the subject faces at the time when the subject steps on the front surface of the biological information measurement device 1. The manner in which the subject steps on the front surface of the biological information measurement device 1 includes an arrangement of the feet of the subject on the front surface of the biological information measurement device 1.

Measurement items of biological information of the subject may be selected depending on an order in which the left foot and the right foot of the subject are placed on the front surface of the biological information measurement device 1. In the case of performing only weight measurement, when a dominant foot is the right foot, it is expected that the subject unintentionally steps on the biological information measurement device 1 from the right foot first, and therefore, for example, settings (1-1) and (1-2) below may be set in the biological information measurement device 1. The following settings (1-1) and (1-2) are examples, and other settings may be set in the biological information measurement device 1.

(1-1) When the left foot of the subject is placed on the front surface of the biological information measurement device 1 and then the right foot of the subject is placed on the front surface of the biological information measurement device 1, the weight, body composition, standing position balance, and cardiovascular condition of the subject are measured.

(1-2) When the right foot of the subject is placed on the front surface of the biological information measurement device 1 and then the left foot of the subject is placed on the front surface of the biological information measurement device 1, the weight and body composition of the subject are measured.

In addition, the subject may have a grasp of settings (1-3) and (1-4) below. The following settings (1-3) and (1-4) are examples, and the subject may have a grasp of other settings.

(1-3) When measuring weight, body composition, standing position balance, and cardiovascular condition, the subject places the left foot on the front surface of the biological information measurement device 1, and then places the right foot on the front surface of the biological information measurement device 1.

(1-4) When measuring weight and body composition, the subject places the right foot on the front surface of the biological information measurement device 1, and then places the left foot on the front surface of the biological information measurement device 1.

The subject may set at least one of the plurality of measurement items in the biological information measurement device 1 by operating the operation unit 11. In addition, the subject may set at least one of the plurality of measurement items in the biological information measurement device 1 by operating the information processing device. For example, as measurement items for a case where the subject places the left foot on the front surface of the biological information measurement device 1 and then places the right foot on the front surface of the biological information measurement device 1, the measurement items of weight, body composition, standing position balance, and cardiovascular condition may be set in the biological information measurement device 1. For example, as measurement items for a case where the right foot is placed on the front surface of the biological information measurement device 1 and then the left foot is placed on the front surface of the biological information measurement device 1, the measurement items of weight and body composition may be set in the biological information measurement device 1.

The order detection unit 51 detects an order in which the left foot and the right foot of the subject are placed on the front surface of the biological information measurement device 1, and outputs the detection result. The measurement unit 42 may select at least one measurement item from among a plurality of measurement items related to the living body of the subject, based on the detection result of the order detection unit 51, and measure the biological information of the subject. The plurality of measurement items related to the living body of the subject include a measurement item for measuring weight, a measurement item for measuring body composition, a measurement item for measuring standing position balance, and a measurement item for measuring cardiovascular condition.

The detection processing by the order detection unit 51 will be described. The order detection unit 51 may detect (determine) an order in which the left foot and the right foot of the subject are placed on the front surface of the biological information measurement device 1, based on the measurement value output from the electrode unit 21. A plurality of electrodes may be disposed on each of the left side and the right side of the front surface of the biological information measurement device 1 and resistance values between the electrodes may be measured. For example, a resistance value between two electrodes disposed on the left side of the front surface of the biological information measurement device 1 is detected (measured), and then a resistance value between two electrodes disposed on the right side of the front surface of the biological information measurement device 1 is detected. In this case, the order detection unit 51 detects that the left foot of the subject is placed on the front surface of the biological information measurement device 1 and then the right foot of the subject is placed on the front surface of the biological information measurement device 1. For example, a resistance value between two electrodes disposed on the right side of the front surface of the biological information measurement device 1 is detected, and then a resistance value between two electrodes disposed on the left side of the front surface of the biological information measurement device 1 is detected. In this case, the order detection unit 51 detects that the right foot of the subject is placed on the front surface of the biological information measurement device 1 and then the left foot of the subject is placed on the front surface of the biological information measurement device 1. The two electrodes disposed on the left side of the front surface of the biological information measurement device 1 may be the electrodes 12 and 14. The two electrodes disposed on the right side of the front surface of the biological information measurement device 1 may be the electrodes 13 and 15.

The order detection unit 51 may detect an order in which the left foot and the right foot of the subject are placed on the front surface of the biological information measurement device 1, based on the measurement value output from the first sensor unit 23. A photoelectric sensor may be disposed on each of the left side and the right side of the front surface of the biological information measurement device 1, and an amount of light received by the photoelectric sensors may be measured.

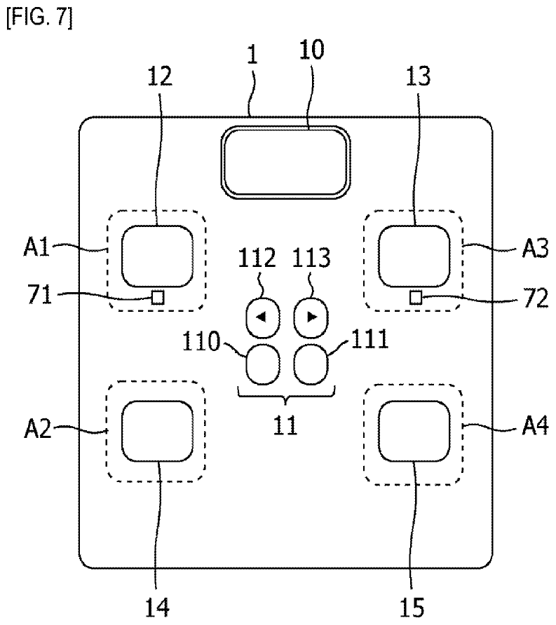

FIG. 7 is a diagram illustrating an arrangement of photoelectric sensors. In the example illustrated in FIG. 7, a photoelectric sensor 71 is disposed between the electrode 12 and the electrode 14, and a photoelectric sensor 72 is disposed between the electrode 13 and the electrode 15. The photoelectric sensors 71 and 72 each include a light emitting diode (LED) that is a light emitting unit that generates light, and a photodiode that is a light reception unit that receives light. The first sensor unit 23 may include the photoelectric sensors 71 and 72. The photoelectric sensor 71 is preferably disposed in the vicinity of the electrode 12 such that the photoelectric sensor 71 reliably comes into contact with the sole of the left foot of the subject when the subject steps on the front surface of the biological information measurement device 1. For example, the photoelectric sensor 71 may be disposed in the vicinity of the electrode 12 such that the photoelectric sensor 71 comes into contact with an underside of the big toe of the left foot of the subject. The photoelectric sensor 71 may be disposed in a region between a dotted line A1 in FIG. 7 and the electrode 12. The photoelectric sensor 71 may be disposed in the vicinity of the electrode 14. The photoelectric sensor 71 may be disposed in a region between a dotted line A2 in FIG. 7 and the electrode 14.

The photoelectric sensor 72 may be disposed in the vicinity of the electrode 13. The photoelectric sensor 72 may be disposed in a region between a dotted line A3 in FIG. 7 and the electrode 13. The photoelectric sensor 72 may be disposed in the vicinity of the electrode 15. The photoelectric sensor 72 may be disposed in a region between a dotted line A4 in FIG. 7 and the electrode 15.

When the left foot of the subject is placed on the front surface of the biological information measurement device 1, a measurement value (an amount of light or a voltage value) output from the photoelectric sensor 71 changes. For example, after the measurement value output from the photoelectric sensor 71 changes, the measurement value output from the photoelectric sensor 72 changes. The order detection unit 51 detects that the left foot of the subject is placed on the front surface of the biological information measurement device 1 and then the right foot of the subject is placed on the front surface of the biological information measurement device 1. In this case, the detection result indicating that the left foot of the subject is placed on the front surface of the biological information measurement device 1 and then the right foot of the subject is placed on the front surface of the biological information measurement device 1 is output from the order detection unit 51. The measurement unit 42 may select all measurement items from among the plurality of measurement items related to the living body of the subject and measure the biological information of the subject.

For example, after the measurement value output from the photoelectric sensor 72 changes, the measurement value output from the photoelectric sensor 71 changes. In this case, the order detection unit 51 detects that the right foot of the subject is placed on the front surface of the biological information measurement device 1 and then the left foot of the subject is placed on the front surface of the biological information measurement device 1.

The order detection unit 51 may detect an order in which the left foot and the right foot of the subject are placed on the front surface of the biological information measurement device 1, based on the measurement values output from the load cells 31 to 34 of the second sensor unit 24. For example, after the measurement value output from the load cell 31 or 33 is detected, the measurement value output from the load cell 32 or 34 is detected. In this case, the order detection unit 51 detects that the left foot of the subject is placed on the front surface of the biological information measurement device 1 and then the right foot of the subject is placed on the front surface of the biological information measurement device 1. For example, after the measurement value output from the load cell 32 or 34 is detected, the measurement value output from the load cell 31 or 33 is detected. In this case, the order detection unit 51 detects that the right foot of the subject is placed on the front surface of the biological information measurement device 1 and then the left foot of the subject is placed on the front surface of the biological information measurement device 1.

When the detection result of the order detection unit 51 is a result indicating that the right foot of the subject is placed on the front surface of the biological information measurement device 1 and then the left foot of the subject is placed thereon, the measurement unit 42 may select a measurement item for measuring weight and a measurement item for measuring body composition from among the plurality of measurement items related to the living body of the subject, and measure the biological information of the subject. In addition, the measurement unit 42 may perform the following processing instead of this processing. The measurement unit 42 may select a measurement item for measuring weight from among the plurality of measurement items related to the living body of the subject and measure the biological information of the subject. Since only the weight of the subject is measured and no measurement is performed for the other measurement items, the time from the start to the completion of the measurement of the biological information of the subject is shortened. The measurement unit 42 may select a measurement item for measuring weight and a measurement item for measuring standing position balance from among the plurality of measurement items related to the living body of the subject and measure the biological information of the subject. Since the weight and the standing position balance of the subject are measured and no measurement is performed for the other measurement items, the time from the start to the completion of the measurement of the biological information of the subject is shortened. The measurement unit 42 may select a measurement item for measuring weight, a measurement item for measuring body composition, and a measurement item for measuring standing position balance from among the plurality of measurement items related to the living body of the subject and measure the biological information of the subject. Since the weight, the body composition, and the standing position balance of the subject are measured and no measurement is performed for the other measurement items, the time from the start to the completion of the measurement of the biological information of the subject is shortened.

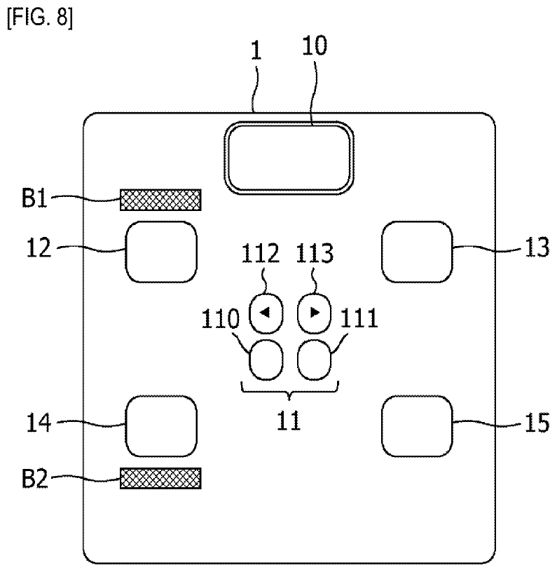

A mark for guiding an order in which the left foot and the right foot of the subject ride are placed on the front surface of the biological information measurement device 1 may be provided on the front surface of the biological information measurement device 1. FIG. 8 is a diagram illustrating a schematic configuration of the biological information measurement device 1 according to an embodiment. In FIG. 8, lighting units such as LEDs are provided in regions B1 and B2 on the front surface of the biological information measurement device 1. The respective lighting units provided in the regions B1 and B2 on the front surface of the biological information measurement device 1 can be turned on, blinked, and turned off. The respective lighting units provided in the regions B1 and B2 on the front surface of the biological information measurement device 1 may be turned on or blinked to guide the subject to intentionally place the left foot on the front surface of the biological information measurement device 1. This leads to an improvement in recognition that a measurement target can be selected and to habituation by placing the left foot first on the front surface of the biological information measurement device 1. Instead of providing the lighting units such as LEDs, marks having a predetermined shape may be provided in the regions B1 and B2 on the front surface of the biological information measurement device 1. When the biological information measurement device 1 has a function for linking with a smartphone carried by the subject during measurement, each lighting unit may be turned on or blinked in a state appropriate for the subject according to a measurement item set in advance on the smartphone by the subject as a target for focused management or a measurement item automatically selected from a measurement tendency over a predetermined period.

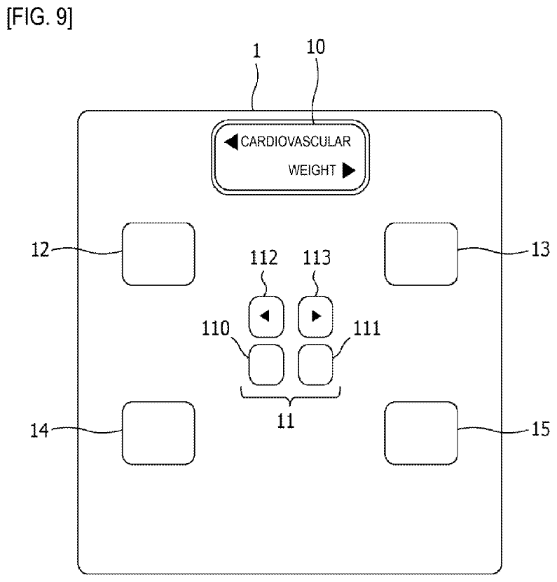

FIG. 9 is a diagram illustrating a schematic configuration of the biological information measurement device 1 according to an embodiment. As illustrated in FIG. 9, the display unit 10 may display at least one measurement item among a plurality of measurement items related to the living body of the subject, as well as a diagram for guiding an order in which the left foot and the right foot of the subject are placed on the front surface of the biological information measurement device 1. In addition, the display unit 10 may display a message for guiding an order in which the left foot and the right foot of the subject are placed on the front surface of the biological information measurement device 1.

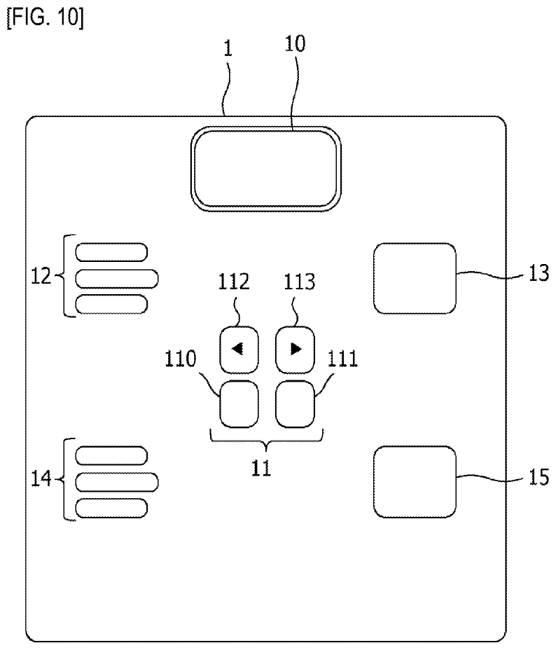

FIG. 10 is a diagram illustrating a schematic configuration of the biological information measurement device 1 according to an embodiment. The electrode 12 is an electrode (first electrode) that can come into contact with the sole of one (left foot) of the feet of the subject, and is configured by a plurality of electrodes. The electrode 14 is an electrode that can come into contact with the sole of one (left foot) of the feet of the subject, and is configured by a plurality of electrodes. The electrode 13 is an electrode that can come into contact with the sole of the other (right foot) of the feet of the subject, and is configured by a single electrode. The electrode 15 is an electrode (second electrode) that can come into contact with the sole of the other (right foot) of the feet of the subject, and is configured by a single electrode. As illustrated in FIG. 10, the number of electrodes (first electrodes) that can come into contact with the sole of one (left foot) of the feet of the subject may differ from the number of electrodes (second electrodes) that can come into contact with the sole of the other (right foot) of the feet of the subject. For example, as illustrated in FIG. 10, the number of first electrodes may be larger than the number of second electrodes so that the subject may be guided to intentionally place the left foot on the front surface of the biological information measurement device 1. As a result, when measuring weight, body composition, standing position balance, and cardiovascular condition, the subject considers it preferable to place the left foot first on the front surface of the biological information measurement device 1. Therefore, the subject can easily distinguish a difference depending on the order in which the feet are placed on the front surface of the biological information measurement device 1, which leads to an improvement in recognition that a measurement target can be selected and to habituation.

Without being limited to the example illustrated in FIG. 10, a shape of the electrode (first electrode) that can come into contact with the sole of one (left foot) of the feet of the subject may differ from a shape of the electrode (second electrode) that can come into contact with the sole of the other (right foot) of the feet of the subject. For example, the first electrode may have a characteristic shape so that the subject may be guided to intentionally place the left foot on the front surface of the biological information measurement device 1.

Measurement items of biological information of the subject may be selected depending on a direction in which the subject faces at the time when the subject steps on the front surface of the biological information measurement device 1. For example, the following settings (2-1) and (2-2) are set in the biological information measurement device 1. The following settings (2-1) and (2-2) are examples, and other settings may be set in the biological information measurement device 1.

(2-1) When the subject steps on the front surface of the biological information measurement device 1 while facing a first direction, the weight of the subject is measured. The first direction is a direction in which the display unit 10 is disposed.

(2-2) When the subject steps on the front surface of the biological information measurement device 1 while facing a second direction, the weight, body composition, standing position balance, and cardiovascular condition of the subject are measured. The second direction is a direction different from the direction in which the display unit 10 is disposed.

Since the measurement of standing position balance and cardiovascular condition is easily affected by body motion of the subject caused by the subject moving the head so as to look at the display of measurement progress, it is preferable that the display unit 10 is configured so as not to be visible to the subject. Therefore, it is preferable to set the above setting (2-2) in the biological information measurement device 1.

In addition, the subject may have a grasp of settings (2-3), (2-4), and (2-5) below. The following settings (2-3), (2-4), and (2-5) are examples, and the subject may have a grasp of other settings.

(2-3) When measuring weight, the subject steps on the front surface of the biological information measurement device 1 while facing the first direction.

(2-4) When measuring weight, body composition, standing position balance, and cardiovascular condition, the subject steps on the front surface of the biological information measurement device 1 while facing the second direction.

(2-5) When the subject steps on the front surface of the biological information measurement device 1, the subject brings the heel side of the foot into contact with the front surface of the biological information measurement device 1, and then brings the toe side of the foot into contact with the front surface of the biological information measurement device 1.

The subject may set at least one of the plurality of measurement items in the biological information measurement device 1 by operating the operation unit 11. In addition, the subject may set at least one of the plurality of measurement items in the biological information measurement device 1 by operating the information processing device. For example, as measurement items for a case where the subject steps on the front surface of the biological information measurement device 1 while facing the first direction, the measurement item of weight may be set in the biological information measurement device 1. For example, as measurement items for a case where the subject steps on the front surface of the biological information measurement device 1 while facing the second direction, the measurement items of weight, body composition, standing position balance, and cardiovascular condition may be set in the biological information measurement device 1.

The direction detection unit 52 detects a direction in which the subject faces at the time when the subject steps on the front surface of the biological information measurement device 1, and outputs the detection result. The measurement unit 42 may select at least one measurement item from among a plurality of measurement items related to the living body of the subject, based on the detection result of the direction detection unit 52, and measure the biological information of the subject.

The detection processing by the direction detection unit 52 will be described. The direction detection unit 52 may detect (determine) a direction in which the subject faces at the time when the subject steps on the front surface of the biological information measurement device 1, based on the measurement value output from the electrode unit 21. The direction detection unit 52 may detect a direction in which the subject faces at the time when the subject steps on the front surface of the biological information measurement device 1 by disposing a plurality of electrodes on the front surface of the biological information measurement device 1 and measuring resistance values between the respective electrodes. The electrode unit 21 may include a plurality of electrodes. The electrodes 12 to 15 may be used.

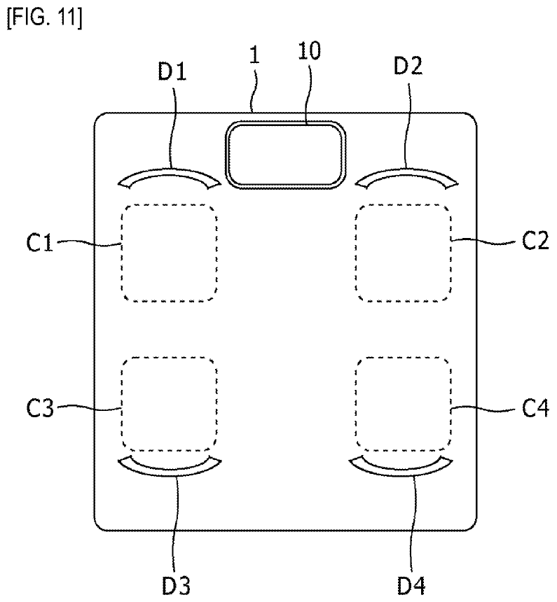

FIGS. 11 to 15 are diagrams illustrating schematic configurations of the biological information measurement device 1 according to embodiments. The detection processing by the direction detection unit 52 will be described with reference to FIGS. 11 to 15. In FIGS. 11 to 15, the operation unit 11 and the electrodes 12 to 15 are not illustrated. In FIGS. 11 to 15, it is assumed that the electrode 12 is disposed in a region C1 on the front surface of the biological information measurement device 1, the electrode 13 is disposed in a region C2 on the front surface of the biological information measurement device 1, the electrode 14 is disposed in a region C3 on the front surface of the biological information measurement device 1, and the electrode 15 is disposed in a region C4 on the front surface of the biological information measurement device 1. Additionally, in FIGS. 11 to 13, lighting units such as LEDs are provided in regions D1 to D4 on the front surfaces of the biological information measurement device 1. The respective lighting units provided in the regions D1 to D4 on the front surface of the biological information measurement device 1 can be turned on, blinked, and turned off.

The respective lighting units provided in the regions D3 and D4 on the front surface of the biological information measurement device 1 may be turned on or blinked to guide the subject to intentionally place the heel of the left foot on the region C3 on the front surface of the biological information measurement device 1 and to guide the subject to intentionally place the heel of the right foot on the region C4 on the front surface of the biological information measurement device 1. This leads to an improvement in the recognition that a measurement target can be selected and to habituation by placing the heel of the left foot of the subject first on the region C3 on the front surface of the biological information measurement device 1 and then placing the heel of the right foot of the subject first on the region C4 of the front surface of the biological information measurement device 1.

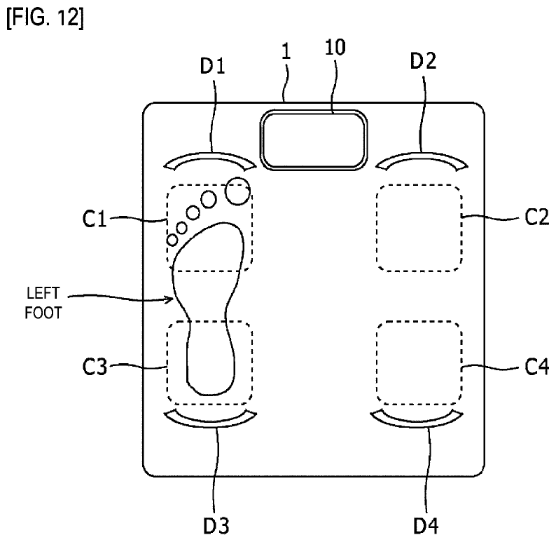

The direction detection unit 52 may detect an order in which the left foot of the subject comes into contact with the regions C1 and C3 on the front surface of the biological information measurement device 1, based on the measurement value output from the electrode unit 21. FIG. 12 schematically illustrates a state in which the left foot of the subject is placed on the front surface of the biological information measurement device 1. For example, a resistance value between two electrodes disposed in the region C3 on the front surface of the biological information measurement device 1 is detected, and then a resistance value between two electrodes disposed in the region C1 on the front surface of the biological information measurement device 1 is detected. In this case, a detection result indicating that the direction in which the subject faces at the time when the subject steps on the front surface of the biological information measurement device 1 is the first direction is output from the direction detection unit 52.

In addition, the direction detection unit 52 may detect an order in which the right foot of the subject comes into contact with the regions C2 and C4 on the front surface of the biological information measurement device 1, based on the measurement value output from the electrode unit 21. For example, a resistance value between two electrodes disposed in the region C4 on the front surface of the biological information measurement device 1 is detected, and then a resistance value between two electrodes disposed in the region C2 on the front surface of the biological information measurement device 1 is detected. In this case, a detection result indicating that the direction in which the subject faces at the time when the subject steps on the front surface of the biological information measurement device 1 is the first direction is output from the direction detection unit 52.

The respective lighting units provided in the regions D1 and D2 on the front surface of the biological information measurement device 1 may be turned on or blinked to guide the subject to intentionally place the heel of the left foot on the region C2 on the front surface of the biological information measurement device 1 and to guide the subject to intentionally place the heel of the right foot on the region C1 on the front surface of the biological information measurement device 1. This leads to an improvement in the recognition that a measurement target can be selected and to habituation by placing the heel of the left foot of the subject first on the region C2 on the front surface of the biological information measurement device 1 and then placing the heel of the right foot of the subject first on the region C1 of the front surface of the biological information measurement device 1.

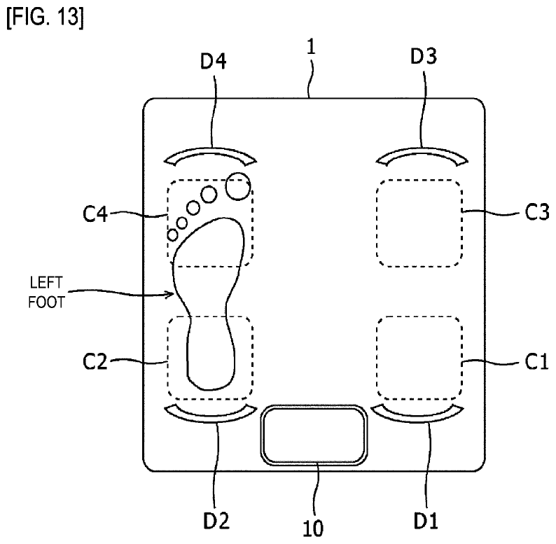

The direction detection unit 52 may detect an order in which the left foot of the subject comes into contact with the regions C2 and C4 on the front surface of the biological information measurement device 1, based on the measurement value output from the electrode unit 21. FIG. 13 schematically illustrates a state in which the left foot of the subject is placed on the front surface of the biological information measurement device 1. For example, a resistance value between two electrodes disposed in the region C2 on the front surface of the biological information measurement device 1 is detected, and then a resistance value between two electrodes disposed in the region C4 on the front surface of the biological information measurement device 1 is detected. In this case, a detection result indicating that the direction in which the subject faces at the time when the subject steps on the front surface of the biological information measurement device 1 is the second direction is output from the direction detection unit 52.

In addition, the direction detection unit 52 may detect an order in which the right foot of the subject comes into contact with the regions C1 and C3 on the front surface of the biological information measurement device 1, based on the measurement value output from the electrode unit 21. For example, a resistance value between two electrodes disposed in the region C1 on the front surface of the biological information measurement device 1 is detected, and then a resistance value between two electrodes disposed in the region C3 on the front surface of the biological information measurement device 1 is detected. In this case, a detection result indicating that the direction in which the subject faces at the time when the subject steps on the front surface of the biological information measurement device 1 is the second direction is output from the direction detection unit 52.

When the direction in which the subject faces at the time when the subject steps on the front surface of the biological information measurement device 1 is the second direction, the electrode function is switched to measure the biological information of the subject. For example, in the example illustrated in FIG. 13, the electrodes 14 and 15 may function as current applying electrodes for applying a constant current to the body of the subject, and the electrodes 12 and 13 may function as voltage measurement electrodes for measuring a voltage during current application.

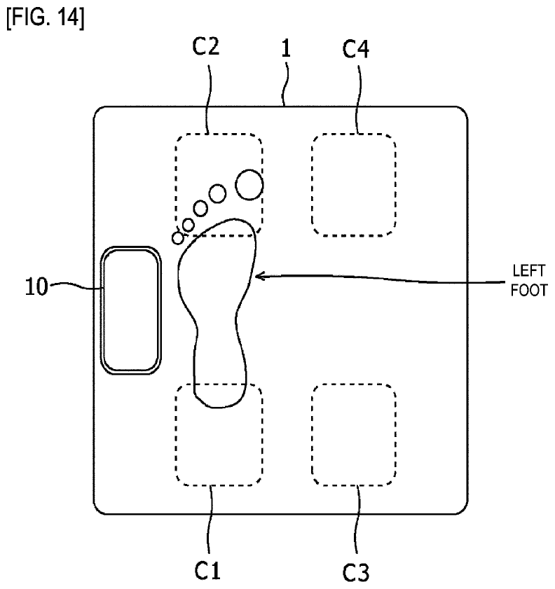

The direction detection unit 52 may detect an order in which the left foot of the subject comes into contact with the regions C1 and C2 on the front surface of the biological information measurement device 1, based on the measurement value output from the electrode unit 21. FIG. 14 schematically illustrates a state in which the left foot of the subject is placed on the front surface of the biological information measurement device 1. For example, a resistance value between two electrodes disposed in the region C1 on the front surface of the biological information measurement device 1 is detected, and then a resistance value between two electrodes disposed in the region C2 on the front surface of the biological information measurement device 1 is detected. In this case, a detection result indicating that the direction in which the subject faces at the time when the subject steps on the front surface of the biological information measurement device 1 is the second direction is output from the direction detection unit 52.

In addition, the direction detection unit 52 may detect an order in which the right foot of the subject comes into contact with the regions C3 and C4 on the front surface of the biological information measurement device 1, based on the measurement value output from the electrode unit 21. For example, a resistance value between two electrodes disposed in the region C3 on the front surface of the biological information measurement device 1 is detected, and then a resistance value between two electrodes disposed in the region C4 on the front surface of the biological information measurement device 1 is detected. In this case, a detection result indicating that the direction in which the subject faces at the time when the subject steps on the front surface of the biological information measurement device 1 is the second direction is output from the direction detection unit 52.

When the direction in which the subject faces at the time when the subject steps on the front surface of the biological information measurement device 1 is the second direction, the electrode function is switched to measure the biological information of the subject. For example, in the example illustrated in FIG. 14, the electrodes 13 and 15 may function as current applying electrodes for applying a constant current to the body of the subject, and the electrodes 12 and 14 may function as voltage measurement electrodes for measuring a voltage during current application.

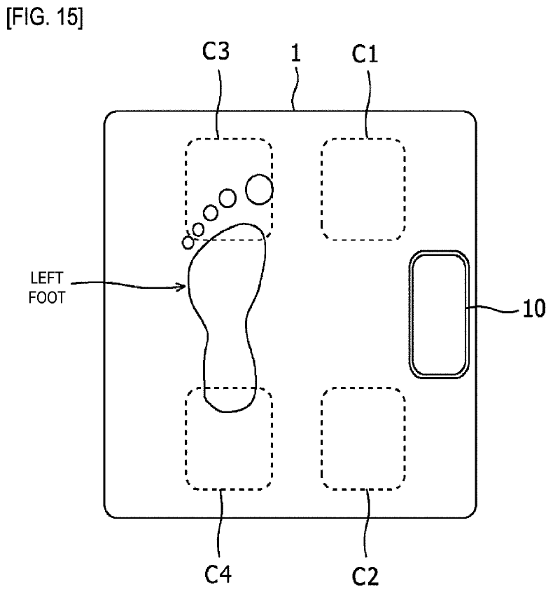

The direction detection unit 52 may detect an order in which the left foot of the subject comes into contact with the regions C3 and C4 on the front surface of the biological information measurement device 1, based on the measurement value output from the electrode unit 21. FIG. 15 schematically illustrates a state in which the left foot of the subject is placed on the front surface of the biological information measurement device 1. For example, a resistance value between two electrodes disposed in the region C4 on the front surface of the biological information measurement device 1 is detected, and then a resistance value between two electrodes disposed in the region C3 on the front surface of the biological information measurement device 1 is detected. In this case, a detection result indicating that the direction in which the subject faces at the time when the subject steps on the front surface of the biological information measurement device 1 is the second direction is output from the direction detection unit 52.

In addition, the direction detection unit 52 may detect an order in which the right foot of the subject comes into contact with the regions C1 and C2 on the front surface of the biological information measurement device 1, based on the measurement value output from the electrode unit 21. For example, a resistance value between two electrodes disposed in the region C2 on the front surface of the biological information measurement device 1 is detected, and then a resistance value between two electrodes disposed in the region C1 on the front surface of the biological information measurement device 1 is detected. In this case, a detection result indicating that the direction in which the subject faces at the time when the subject steps on the front surface of the biological information measurement device 1 is the second direction is output from the direction detection unit 52.

When the direction in which the subject faces at the time when the subject steps on the front surface of the biological information measurement device 1 is the second direction, the electrode function is switched to measure the biological information of the subject. For example, in the example illustrated in FIG. 15, the electrodes 12 and 13 may function as current applying electrodes for applying a constant current to the body of the subject, and the electrodes 14 and 15 may function as voltage measurement electrodes for measuring a voltage during current application.

A heel side of a foot of the subject is drier than a toe side of the foot of the subject, and a resistance value of the heel side of the foot is larger than a resistance value of the toe side of the foot. The direction detection unit 52 may detect a direction in which the subject faces at the time when the subject steps on the front surface of the biological information measurement device 1 by comparing a resistance value on a heel side of a foot of the subject with a resistance value on a toe side of the foot of the subject. When a resistance value between two electrodes disposed in the region C3 on the front surface of the biological information measurement device 1 is larger than a resistance value between two electrodes disposed in the region C1 on the front surface of the biological information measurement device 1, a detection result indicating that the direction in which the subject faces at the time when the subject steps on the front surface of the biological information measurement device 1 is the first direction is output from the direction detection unit 52. When a resistance value between two electrodes disposed in the region C2 on the front surface of the biological information measurement device 1 is larger than a resistance value between two electrodes disposed in the region C4 on the front surface of the biological information measurement device 1, a detection result indicating that the direction in which the subject faces at the time when the subject steps on the front surface of the biological information measurement device 1 is the second direction is output from the direction detection unit 52.

When the detection result of the direction detection unit 52 is a result indicating that the right foot of the subject is placed on the front surface of the biological information measurement device 1 and then the left foot of the subject is placed thereon, the measurement unit 42 may select a measurement item for measuring weight and a measurement item for measuring body composition from among the plurality of measurement items related to the living body of the subject, and measure the biological information of the subject. In addition, the measurement unit 42 may perform the following processing instead of this processing. The measurement unit 42 may select a measurement item for measuring weight from among the plurality of measurement items related to the living body of the subject and measure the biological information of the subject. Since only the weight of the subject is measured and no measurement is performed for the other measurement items, the time from the start to the completion of the measurement of the biological information of the subject is shortened. The measurement unit 42 may select a measurement item for measuring weight and a measurement item for measuring standing position balance from among the plurality of measurement items related to the living body of the subject and measure the biological information of the subject. Since the weight and the standing position balance of the subject are measured and no measurement is performed for the other measurement items, the time from the start to the completion of the measurement of the biological information of the subject is shortened. The measurement unit 42 may select a measurement item for measuring weight, a measurement item for measuring body composition, and a measurement item for measuring standing position balance from among the plurality of measurement items related to the living body of the subject and measure the biological information of the subject. Since the weight, the body composition, and the standing position balance of the subject are measured and no measurement is performed for the other measurement items, the time from the start to the completion of the measurement of the biological information of the subject is shortened.

The direction detection unit 52 may detect an order in which the left foot of the subject comes into contact with the regions C1 and C3 on the front surface of the biological information measurement device 1, based on the measurement value output from the first sensor unit 23. A photoelectric sensor may be disposed in each of the regions C1 and C3 on the front surface of the biological information measurement device 1, and an amount of light received by the photoelectric sensors may be measured. The direction detection unit 52 may detect an order in which the right foot of the subject comes into contact with the regions C2 and C4 on the front surface of the biological information measurement device 1, based on the measurement value output from the first sensor unit 23. A photoelectric sensor may be disposed in each of the regions C2 and C4 on the front surface of the biological information measurement device 1, and an amount of light received by the photoelectric sensors may be measured.

The direction detection unit 52 may detect an order in which the left foot of the subject comes into contact with the regions C2 and C4 on the front surface of the biological information measurement device 1, based on the measurement value output from the first sensor unit 23. A photoelectric sensor may be disposed in each of the regions C2 and C4 on the front surface of the biological information measurement device 1, and an amount of light received by the photoelectric sensors may be measured. The direction detection unit 52 may detect an order in which the right foot of the subject comes into contact with the regions C1 and C3 on the front surface of the biological information measurement device 1, based on the measurement value output from the first sensor unit 23. A photoelectric sensor may be disposed in each of the regions C1 and C3 on the front surface of the biological information measurement device 1, and an amount of light received by the photoelectric sensors may be measured.

The direction detection unit 52 may detect an order in which the left foot of the subject comes into contact with the regions C1 and C2 on the front surface of the biological information measurement device 1, based on the measurement value output from the first sensor unit 23. A photoelectric sensor may be disposed in each of the regions C1 and C2 on the front surface of the biological information measurement device 1, and an amount of light received by the photoelectric sensors may be measured. The direction detection unit 52 may detect an order in which the right foot of the subject comes into contact with the regions C3 and C4 on the front surface of the biological information measurement device 1, based on the measurement value output from the first sensor unit 23. A photoelectric sensor may be disposed in each of the regions C3 and C4 on the front surface of the biological information measurement device 1, and an amount of light received by the photoelectric sensors may be measured.

The direction detection unit 52 may detect an order in which the left foot of the subject comes into contact with the regions C3 and C4 on the front surface of the biological information measurement device 1, based on the measurement value output from the first sensor unit 23. A photoelectric sensor may be disposed in each of the regions C3 and C4 on the front surface of the biological information measurement device 1, and an amount of light received by the photoelectric sensors may be measured. The direction detection unit 52 may detect an order in which the right foot of the subject comes into contact with the regions C1 and C2 on the front surface of the biological information measurement device 1, based on the measurement value output from the first sensor unit 23. A photoelectric sensor may be disposed in each of the regions C1 and C2 on the front surface of the biological information measurement device 1, and an amount of light received by the photoelectric sensors may be measured.

The direction detection unit 52 may detect an order in which the left foot of the subject comes into contact with the regions C1 and C3 on the front surface of the biological information measurement device 1, based on the measurement values output from the load cells 31 and 33 of the second sensor unit 24. The direction detection unit 52 may detect an order in which the right foot of the subject comes into contact with the regions C2 and C4 on the front surface of the biological information measurement device 1, based on the measurement values output from the load cells 32 and 34 of the second sensor unit 24.

The direction detection unit 52 may detect an order in which the left foot of the subject comes into contact with the regions C2 and C4 on the front surface of the biological information measurement device 1, based on the measurement values output from the load cells 32 and 34 of the second sensor unit 24. The direction detection unit 52 may detect an order in which the right foot of the subject comes into contact with the regions C1 and C3 on the front surface of the biological information measurement device 1, based on the measurement values output from the load cells 31 and 33 of the second sensor unit 24.

The direction detection unit 52 may detect an order in which the left foot of the subject comes into contact with the regions C1 and C2 on the front surface of the biological information measurement device 1, based on the measurement values output from the load cells 31 and 32 of the second sensor unit 24. The direction detection unit 52 may detect an order in which the right foot of the subject comes into contact with the regions C3 and C4 on the front surface of the biological information measurement device 1, based on the measurement values output from the load cells 33 and 34 of the second sensor unit 24.

The direction detection unit 52 may detect an order in which the left foot of the subject comes into contact with the regions C3 and C4 on the front surface of the biological information measurement device 1, based on the measurement values output from the load cells 33 and 34 of the second sensor unit 24. The direction detection unit 52 may detect an order in which the right foot of the subject comes into contact with the regions C1 and C2 on the front surface of the biological information measurement device 1, based on the measurement values output from the load cells 31 and 32 of the second sensor unit 24.

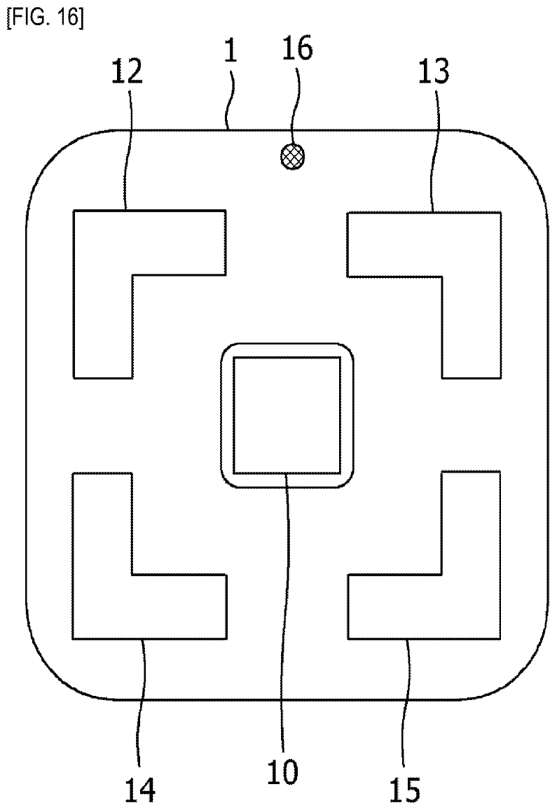

FIG. 16 is a diagram illustrating a schematic configuration of the biological information measurement device 1 according to an embodiment. The biological information measurement device 1 illustrated in FIG. 16 includes a display unit 10 and L-shaped electrodes 12 to 15. The biological information measurement device 1 has a substantially symmetrical appearance in upper-lower and left-right directions. That is, the biological information measurement device 1 has the same vertical and horizontal dimensions and has an appearance that does not give a sense of directionality. The operation unit is integrated with the display unit 10, and is, for example, a touch panel. The display unit 10 is provided at a center portion of the front surface of the biological information measurement device 1. The display unit 10 is disposed at the center portion of each diagonal line of the electrodes 12 to 15. According to the biological information measurement device 1 illustrated in FIG. 16, the soles of the feet of the subject reliably come into contact with the electrodes 12 to 15 regardless of the direction in which the subject steps on the front surface of the biological information measurement device 1. The display unit 10 changes a display orientation of a measurement result, data, and information depending on the direction in which the subject faces at the time when the subject steps on the front surface of the biological information measurement device 1, and displays the measurement result, the information, and the data.

As illustrated in FIG. 16, a lighting unit 16 is provided on the front surface of the biological information measurement device 1. The lighting unit 16 can be turned on, blinked, and turned off. When the lighting unit 16 is turned on or blinked, the subject steps on the front surface of the biological information measurement device 1 so as to face a first direction or a second direction with reference to the lighting unit 16. The first direction is a direction in which the lighting unit 16 is disposed. The second direction is a direction different from the direction in which the lighting unit 16 is disposed.

The biological information measurement device 1 can be incorporated into a floor of a building such as a house. An example in which the biological information measurement device 1 is incorporated into a floor will be described. In the biological information measurement device 1 of a type incorporated into the floor, the first direction may be a direction in which a mirror is disposed. When measuring weight, the subject steps on the front surface of the biological information measurement device 1 while facing the first direction. As a result, the weight of the subject can be measured while the subject looks at the mirror or while the subject also looks at the body shape of the subject reflected in the mirror.

In addition, in the biological information measurement device 1 of the type incorporated into the floor, the second direction may be a direction in which the subject faces when the subject's back is against a wall of a building. When measuring weight, body composition, standing position balance, and cardiovascular condition, the subject steps on the front surface of the biological information measurement device 1 while facing the second direction. As a result, the weight, body composition, standing position balance, and cardiovascular condition of the subject can be measured while the subject's back is against a wall of a building. The subject can use a wall of a building to stabilize posture.

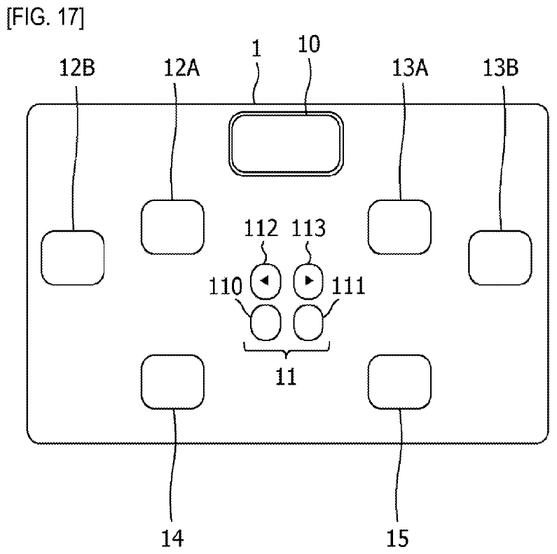

FIGS. 17 to 19 are diagrams illustrating schematic configurations of the biological information measurement device 1 according to an embodiment. The detection processing by the arrangement detection unit 53 will be described with reference to FIGS. 17 to 19. The biological information measurement device 1 illustrated in FIGS. 17 to 19 includes a display unit 10, an operation unit 11, and electrodes 12A, 12B, 13A, 13B, 14, and 15.

Measurement items of biological information of the subject may be selected depending on an arrangement of the feet of the subject on the front surface of the biological information measurement device 1. For example, the following settings (3-1) and (3-2) may be set in the biological information measurement device 1. The following settings (3-1) and (3-2) are examples, and other settings may be set in the biological information measurement device 1.

(3-1) When an arrangement of the feet of the subject is a first arrangement, the weight, body composition, standing position balance, and cardiovascular condition of the subject are measured. The first arrangement is an arrangement of the feet of the subject when the subject steps on the front surface of the biological information measurement device 1 with an interval between the toe side of the left foot and the toe side of the right foot of the subject widened.

(3-2) When the arrangement of the feet of the subject is a second arrangement, the weight and body composition of the subject are measured. The second arrangement is an arrangement of the feet of the subject when the subject steps on the front surface of the biological information measurement device 1 with the left foot and the right foot of the subject parallel to each other.

In addition, the subject may have a grasp of settings (3-3) and (3-4) below. The following settings (3-3) and (3-4) are examples, and the subject may have a grasp of other settings.

(3-3) When measuring weight, body composition, standing position balance, and cardiovascular condition, the subject places the left foot and the right foot on the front surface of the biological information measurement device 1 with the interval between the toe side of the left foot and the toe side of the right foot widened.

(3-4) When measuring weight and body composition, the subject places the left foot and the right foot on the front surface of the biological information measurement device 1 with the left foot and the right foot parallel to each other.

The subject may set at least one of the plurality of measurement items in the biological information measurement device 1 by operating the operation unit 11. In addition, the subject may set at least one of the plurality of measurement items in the biological information measurement device 1 by operating the information processing device. For example, as measurement items for a case where the subject places the left foot and the right foot on the front surface of the biological information measurement device 1 with the interval between the toe side of the left foot and the toe side of the right foot widened, the measurement items of weight, body composition, standing position balance, and cardiovascular condition may be set in the biological information measurement device 1. For example, as measurement items for a case where the subject places the left foot and the right foot on the front surface of the biological information measurement device 1 with the left foot and the right foot parallel to each other, the measurement items of weight and body composition may be set in the biological information measurement device 1.

The arrangement detection unit 53 may detect (determine) an arrangement of the feet of the subject on the front subject of the biological information measurement device 1, based on the measurement value output from the electrode unit 21. The arrangement detection unit 53 may detect (determine) an arrangement of the feet of the subject on the front surface of the biological information measurement device 1 by specifying, among the plurality of electrodes disposed on the front surface of the biological information measurement device 1, two electrodes that are in a conductive state due to contact with the feet of the subject.

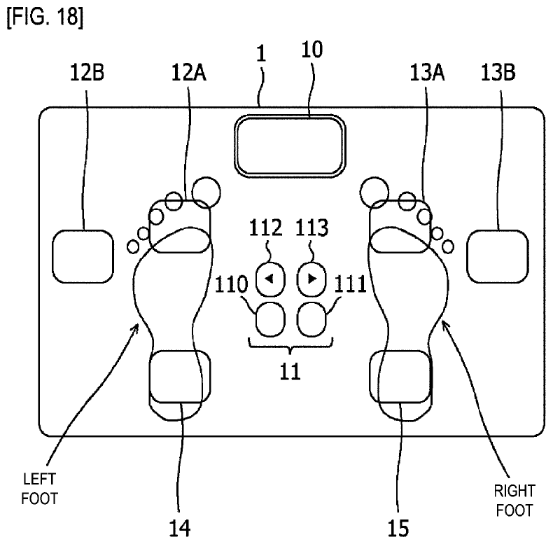

FIG. 18 schematically illustrates a state in which the left foot and the right foot of the subject are placed on the front surface of the biological information measurement device 1. Since the left foot of the subject is in contact with the electrodes 12A and 14, the electrodes 12A and 14 are in a conductive state. Since the right foot of the subject is in contact with the electrodes 13A and 15, the electrodes 13A and 15 are in a conductive state. The arrangement detection unit 53 determines that the arrangement of the feet of the subject on the front surface of the biological information measurement device 1 is a first arrangement. In this case, a detection result indicating that the arrangement of the feet of the subject on the front surface of the biological information measurement device 1 is a first arrangement is output from the arrangement detection unit 53.

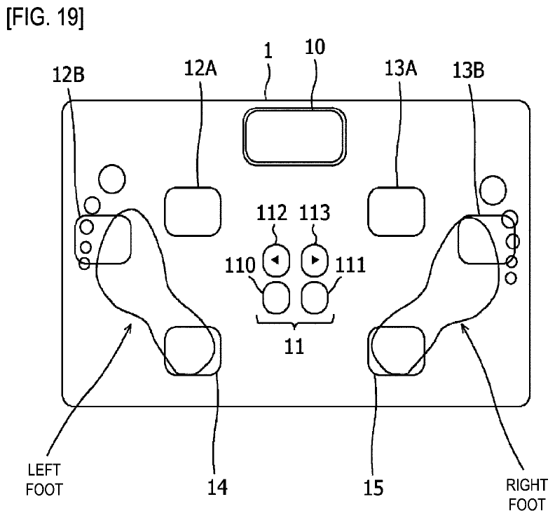

FIG. 19 schematically illustrates a state in which the left foot and the right foot of the subject are placed on the front surface of the biological information measurement device 1. Since the left foot of the subject is in contact with the electrodes 12B and 14, the electrodes 12B and 14 are in a conductive state. Since the right foot of the subject is in contact with the electrodes 13B and 15, the electrodes 13B and 15 are in a conductive state. The arrangement detection unit 53 determines that the arrangement of the feet of the subject on the front surface of the biological information measurement device 1 is a second arrangement. In this case, a detection result indicating that the arrangement of the feet of the subject on the front surface of the biological information measurement device 1 is a second arrangement is output from the arrangement detection unit 53.

The arrangement detection unit 53 detects the arrangement of the feet of the subject on the front surface of the biological information measurement device 1, and outputs the detection result. The measurement unit 42 may select at least one measurement item from among a plurality of measurement items related to the living body of the subject, based on the detection result of the arrangement detection unit 53, and measure the biological information of the subject.

An example in which the above settings (3-1) and (3-2) are set in the biological information measurement device 1 will be described. When the detection result of the arrangement detection unit 53 is a result indicating that the arrangement of the feet of the subject is a first arrangement, the measurement unit 42 may select all measurement items from among the plurality of measurement items related to the living body of the subject and measure the biological information of the subject.

When the detection result of the arrangement detection unit 53 is a result indicating that the arrangement of the feet of the subject is a second arrangement, the measurement unit 42 may select a measurement item for measuring weight and a measurement item for measuring body composition from among the plurality of measurement items related to the living body of the subject and measure the biological information of the subject. In addition, the measurement unit 42 may perform the following processing instead of this processing. The measurement unit 42 may select a measurement item for measuring weight from among the plurality of measurement items related to the living body of the subject and measure the biological information of the subject. Since only the weight of the subject is measured and no measurement is performed for the other measurement items, the time from the start to the completion of the measurement of the biological information of the subject is shortened. The measurement unit 42 may select a measurement item for measuring weight and a measurement item for measuring standing position balance from among the plurality of measurement items related to the living body of the subject and measure the biological information of the subject. Since the weight and the standing position balance of the subject are measured and no measurement is performed for the other measurement items, the time from the start to the completion of the measurement of the biological information of the subject is shortened. The measurement unit 42 may select a measurement item for measuring weight, a measurement item for measuring body composition, and a measurement item for measuring standing position balance from among the plurality of measurement items related to the living body of the subject and measure the biological information of the subject. Since the weight, the body composition, and the standing position balance of the subject are measured and no measurement is performed for the other measurement items, the time from the start to the completion of the measurement of the biological information of the subject is shortened.

The following settings (4-1) and (4-2) may be set in the biological information measurement device 1, instead of the above settings (3-1) and (3-2). The following settings (4-1) and (4-2) are examples, and other settings may be set in the biological information measurement device 1.

(4-1) When the arrangement of the feet of the subject is the first arrangement, the weight of the subject is measured.

(4-2) When the arrangement of the feet of the subject is the second arrangement, the weight, body composition, standing position balance, and cardiovascular condition of the subject are measured.

The subject may have a grasp of the following settings (4-3) and (4-4) instead of the above settings (3-3) and (3-4). The following settings (4-3) and (4-4) are examples, and the subject may have a grasp of other settings.

(4-3) When measuring weight, the subject places the left foot and the right foot on the front surface of the biological information measurement device 1 with the interval between the toe side of the left foot and the toe side of the right foot widened.

(4-4) When measuring weight, body composition, standing position balance, and cardiovascular condition, the subject places the left foot and the right foot on the front surface of the biological information measurement device 1 with the left foot and the right foot parallel to each other.

In the biological information measurement device 1 illustrated in FIGS. 17 to 19, when the subject changes the measurement strategy after stepping on the front surface of the biological information measurement device 1, the subject does not need to move the foot significantly or step on the front surface of the biological information measurement device 1 again. The subject can change the arrangement of the feet by moving the toe by a minimum necessary amount. By providing the electrode 14 with a curved recess portion, the left foot can be easily rotated with the heel of the left foot as a fulcrum. By providing the electrode 15 with a curved recess portion, the right foot can be easily rotated with the heel of the right foot as a fulcrum.

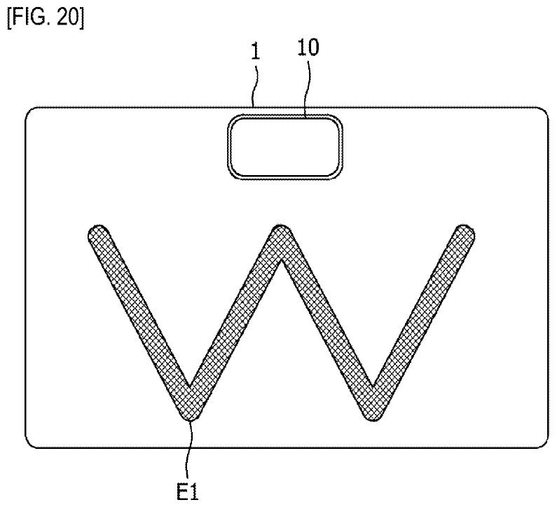

FIG. 20 is a diagram illustrating a schematic configuration of the biological information measurement device 1 according to an embodiment. In FIG. 20, the operation unit 11 and the electrodes 12 to 15 are not illustrated. In FIG. 20, a lighting unit such as an LED is provided in a region E1 on the front surface of the biological information measurement device 1. The lighting unit provided in the region E1 on the front surface of the biological information measurement device 1 can be turned on, blinked, and turned off. The lighting unit provided in the region E1 on the front surface of the biological information measurement device 1 is turned on or blinked, so that a W-shaped (a shape representing Weight) display pattern is displayed on the front surface of the biological information measurement device 1. For example, when the weight of the subject is measured with the interval between the toe side of the left foot and the toe side of the right foot of the subject widened, a W-shaped display pattern is displayed on the front surface of the biological information measurement device 1. This makes it possible to guide the subject to intentionally place the left foot and the right foot on the W-shaped display pattern displayed on the front surface of the biological information measurement device 1.

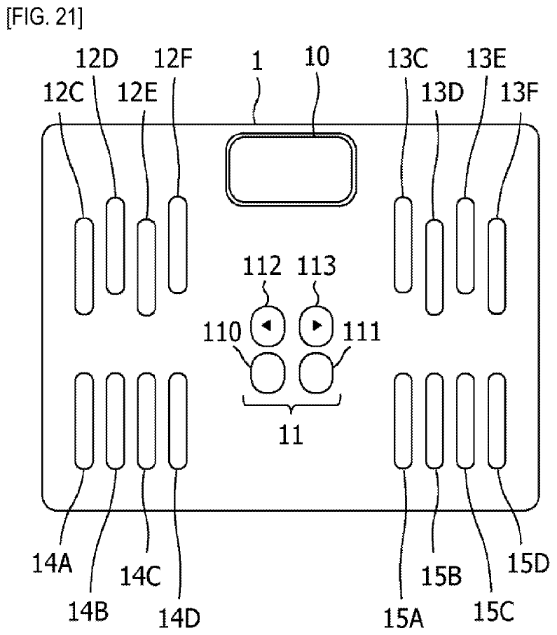

FIG. 21 is a diagram illustrating a schematic configuration of the biological information measurement device 1 according to an embodiment. The biological information measurement device 1 illustrated in FIG. 21 includes the display unit 10, the operation unit 11, and electrodes 12C, 12D, 12E, 12F, 13C, 13D, 13E, 13F, 14A, 14B, 14C, 14D, 15A, 15B, 15C, and 15D. In determining a conductive state when the left foot of the subject is placed on the front surface of the biological information measurement device 1, the electrodes 12C, 12F, and 14A to 14D are used. In determining a conductive state when the right foot of the subject is placed on the front surface of the biological information measurement device 1, the electrodes 13C, 13F, and 15A to 15D are used. With respect to the electrode arrangement of the biological information measurement device 1 illustrated in FIG. 17, by arranging a plurality of elongated electrodes, an increase in size of the device can be suppressed as in the biological information measurement device 1 illustrated in FIG. 21.

A time for measurement of biological information of the subject by the measurement unit 42 may differ depending on the stepping manner detected by the detection unit 41. The measurement time for biological information will be described. A measurement time for biological information (weight and body composition) for measurement items of weight and body composition is longer than a measurement time for biological information (weight) for a measurement item of weight. A measurement time for biological information (weight and standing position balance) for measurement items of weight and standing position balance is longer than the measurement time for biological information (weight). A measurement time for biological information (weight, body composition, standing position balance, and cardiovascular condition) for measurement items of weight, body composition, standing position balance, and cardiovascular condition is longer than the measurement time for biological information (weight and body composition). The measurement time for biological information (weight, body composition, standing position balance, and cardiovascular condition) is longer than the measurement time for biological information (weight and standing position balance). When at least one of the plurality of measurement items is selected depending on the stepping manner detected by the detection unit 41 and the biological information of the subject is measured, a time for measurement of biological information of the subject by the measurement unit 42 differs for each measurement item.

The measurement unit 42 may set a time for measurement of biological information of the subject depending on the stepping manner detected by the detection unit 41. When the measurement item of cardiovascular condition is selected and the biological information of the subject is measured, a first predetermined time may be set as the measurement time for biological information, or a second predetermined time longer than the first predetermined time may be set as the measurement time for biological information. By setting the second predetermined time as the measurement time for biological information, the measurement unit 42 can collect more detailed data than when the first predetermined time is set as the measurement time for biological information.

The measurement unit 42 may set a measurement time for biological information of the subject, based on the detection result of the order detection unit 51. For example, when the subject places the left foot on the front surface of the biological information measurement device 1 and then places the right foot on the front surface of the biological information measurement device 1, the measurement unit 42 may set the first predetermined time as the measurement time for biological information, select the measurement item of cardiovascular condition, and measure the biological information of the subject. For example, when the subject places the right foot on the front surface of the biological information measurement device 1 and then places the left foot on the front surface of the biological information measurement device 1, the measurement unit 42 may set the second predetermined time as the measurement time for biological information, select the measurement item of cardiovascular condition, and measure the biological information of the subject. The measurement unit 42 may set a time for measuring biological information of the subject, based on the detection result of the direction detection unit 52. The measurement unit 42 may set a measurement time for measuring biological information of the subject, based on the detection result of the arrangement detection unit 53.

The measurement result of the biological information measured by the measurement unit 42 is stored in the storage unit 27. The measurement result of the biological information may be transmitted to the information processing device of the subject via the communication unit 26. The measurement result of the biological information may be stored in a storage unit of the information processing device of the subject. A recording target of the measurement result of the biological information may differ depending on the stepping manner detected by the detection unit 41. The measurement unit 42 may determine whether to record the measurement result of the biological information, based on the detection result of the detection unit 41.

The measurement unit 42 may determine whether to record the measurement result, based on the detection result of the order detection unit 51. For example, when the subject places the left foot on the front surface of the biological information measurement device 1 and then places the right foot on the front surface of the biological information measurement device 1, the measurement unit 42 selects the measurement item of weight, measures the biological information of the subject, and records the measurement result of the biological information in the database of the storage unit 27. For example, when the subject places the right foot on the front surface of the biological information measurement device 1 and then places the left foot on the front surface of the biological information measurement device 1, the measurement unit 42 selects the measurement item of weight, measures the biological information of the subject, and does not record the measurement result of the biological information in the database of the storage unit 27. The measurement unit 42 may determine whether to record the measurement result, based on the detection result of the direction detection unit 52. The measurement unit 42 may determine whether to record the measurement result, based on the detection result of the arrangement detection unit 53.

The measurement unit 42 may determine whether to record a measurement result of at least one of the plurality of measurement items, based on the detection result of the order detection unit 51. For example, when the subject places the left foot on the front surface of the biological information measurement device 1 and then places the right foot on the front surface of the biological information measurement device 1, the measurement unit 42 selects the measurement items of weight, body composition, standing position balance, and cardiovascular condition. The measurement unit 42 measures the biological information of the subject, and records the measurement result of biological information of at least one of the measurement items of weight, body composition, standing position balance, and cardiovascular condition in the database of the storage unit 27. For example, when the subject places the right foot on the front surface of the biological information measurement device 1 and then places the left foot on the front surface of the biological information measurement device 1, the measurement unit 42 selects the measurement items of weight, body composition, standing position balance, and cardiovascular condition. The measurement unit 42 measures the biological information of the subject, and does not record the measurement result of biological information of the measurement items of weight, body composition, standing position balance, and cardiovascular condition in the database of the storage unit 27. The measurement unit 42 may determine whether to record the measurement result, based on the detection result of the direction detection unit 52. The measurement unit 42 may determine whether to record the measurement result, based on the detection result of the arrangement detection unit 53.

When the measurement of biological information of the subject is performed for all of the plurality of measurement items related to the living body of the subject, the display unit 10 may not display information while the measurement processing is performed by the measurement unit 42. A motion of the subject looking at the information displayed on the display unit 10 (a motion of looking at the feet) affects the measurement of cardiovascular condition. The display unit 10 does not display information while the measurement processing is performed by the measurement unit 42, and thus body motion of the subject can be suppressed, and the accuracy in measuring cardiovascular condition is improved.

### Computer Readable Recording Medium

A program for causing an information processing device or any other machine or apparatus (hereinafter, referred to as a computer or the like) to realize any of the functions described above can be recorded on a recording medium readable by the computer and the like. Then, the computer or the like is caused to read and execute the program of the recording medium, thereby providing the functions.

Here, the recording medium readable by the computer or the like refers to a recording medium that can store information such as data and programs by an electrical, magnetic, optical, mechanical, or chemical action and can be read from the computer or the like. Among such recording media, those removable from the computer or the like include, for example, a flexible disk, a magneto-optical disk, a CD-ROM, a CD-R/W, a DVD, a Blu-ray disk, a flash memory, and the like. Further, such a recording medium fixed to the computer or the like includes a hard disk, a ROM, and the like.

### REFERENCE SIGNS LIST

1: Biological information measurement device

10: Display unit

11: Operation unit

12, 13: Current applying electrode

14, 15: Voltage measurement electrode

20: Control unit

21: Electrode unit

22: Impedance measurement unit

23: First sensor unit

24: Second sensor unit

25: Temperature measurement unit

26: Communication unit

27: Storage unit

31, 32, 33, 34: Load cell

35: Acceleration sensor

41: Detection unit

42: Measurement unit

51: Order detection unit

52: Direction detection unit

53: Arrangement detection unit

54: Weight measurement unit

55: Body composition measurement unit

56: Standing position balance measurement unit

57: Cardiovascular measurement unit

What is claimed is:

1. A biological information measurement device having a placement surface on which a subject is to step, the biological information measurement device comprising:
a detection unit configured to detect a stepping manner selected by the subject from among a plurality of stepping manners for the placement surface; and
a measurement unit configured to measure biological information of the subject, wherein
an operation of the measurement unit differs depending on the stepping manner detected by the detection unit, and
the detection unit detects the stepping manner selected by the subject by detecting an order in which a left foot and a right foot of the subject are placed on the placement surface or detects the stepping manner selected by the subject by detecting a direction in which the subject faces at the time when the subject steps on the placement surface.

2. The biological information measurement device according to claim 1, wherein the detection unit detects the stepping manner selected by the subject by detecting an arrangement of feet of the subject on the placement surface.

3. The biological information measurement device according to claim 1, wherein a time for measurement of the biological information by the measurement unit differs depending on the stepping manner detected by the detection unit.

4. The biological information measurement device according to claim 2, wherein a time for measurement of the biological information by the measurement unit differs depending on the stepping manner detected by the detection unit.

5. The biological information measurement device according to claim 1, wherein a measurement item for measurement of the biological information by the measurement unit differs depending on the stepping manner detected by the detection unit.

6. The biological information measurement device according to claim 2, wherein a measurement item for measurement of the biological information by the measurement unit differs depending on the stepping manner detected by the detection unit.

7. The biological information measurement device according to claim 1, wherein a recording target of a measurement result of the biological information differs depending on the stepping manner detected by the detection unit.

8. The biological information measurement device according to claim 2, wherein a recording target of a measurement result of the biological information differs depending on the stepping manner detected by the detection unit.

9. The biological information measurement device according to claim 1, further comprising:
a first electrode being to come into contact with a sole of one of both feet of the subject; and
a second electrode being to come into contact with a sole of the other of both feet of the subject, wherein
the number of the first electrodes differs from the number of the second electrodes.

10. The biological information measurement device according to claim 1, further comprising:
a first electrode being to come into contact with a sole of one of both feet of the subject; and
a second electrode being to come into contact with a sole of the other of both feet of the subject, wherein
a shape of the first electrode differs from a shape of the second electrode.

11. A biological information measurement method executable by a computer of a biological information measurement device having a placement surface on which a subject is to step, the method comprising:
a detection step of detecting a stepping manner selected by the subject from among a plurality of stepping manners for the placement surface; and
a measurement step of measuring biological information of the subject, wherein
processing in the measurement step differs depending on the stepping manner detected in the detection step, and
in the detection step, the stepping manner selected by the subject is detected by detecting an order in which a left foot and a right foot of the subject are placed on the placement surface, or the stepping manner selected by the subject is detected by detecting a direction in which the subject faces at the time when the subject steps on the placement surface.

12. A program executable by a computer of a biological information measurement device having a placement surface on which a subject is to step, the program causing the computer to execute:
a detection step of detecting a stepping manner selected by the subject from among a plurality of stepping manners for the placement surface; and
a measurement step of measuring biological information of the subject, wherein
processing in the measurement step differs depending on the stepping manner detected in the detection step, and
in the detection step, the stepping manner selected by the subject is detected by detecting an order in which a left foot and a right foot of the subject are placed on the placement surface, or the stepping manner selected by the subject is detected by detecting a direction in which the subject faces at the time when the subject steps on the placement surface.
